(19) United States
(12) Patent Application Publication
(10) Pub. No.: US 2002/0188961 A1
(43) Pub. Date: Dec. 12, 2002

(54) NON HUMAN TRANSGENIC ANIMAL IN WHICH THE EXPRESSION OF THE GENE CODING FOR INSULIN IS DELETED

(76) Inventors: JACQUES JAMI, PARIS (FR); DANIELLE BUCCHINI, PARIS (FR); RAJIV LOCHAN JOSHI, MASSY (FR)

Correspondence Address: YOUNG & THOMPSON, 745 SOUTH 23RD STREET 2ND FLOOR, ARLINGTON, VA, 22202

(21) Appl. No.: 09/402,316

(22) PCT Filed: Apr. 2, 1998
(86) PCT No.: PCT/FR98/00667

(30) Foreign Application Priority Data
Apr. 4, 1997 (FR) 97/04125

(51) Int. Cl. A01K 67/027

(52) U.S. Cl. 800/3; 800/8; 800/14; 800/21; 800/22

(57) ABSTRACT

The invention concerns the use of a non-human transgenic mammal wherein at least one of the alleles of at least one of two genes coding for endogenous insulin is made functionally inoperative with respect to the expression of insulin for determining medicines acting on pathologies involving insulin.

The invention relates to a non-human transgenic animal in which expression of the gene coding for insulin is suppressed.

The single gene for insulin in man, the two genes for insulin in rats and the two genes for insulin in mice have been cloned for a number of years.

In both mice and rats, the two genes code for very similar functional insulins (insulins 1 and 2 differ only by two amino acids) produced in similar proportions. The sequence of these genes is known; it is thought to be identical in all mouse lines. This is not true of the nucleotide sequences in the DNA regions flanking the gene on either side. To increase the very low probability of recombination at the site of an endogenous gene, it is necessary for the flanking sequences to be as strictly homologous as possible on the recombination vector to be constructed and on the cellular DNA. However, this homology is only strict inside a consanguine line of mice.

It is known that insulin, a peptide hormone secreted by the β cells of the pancreatic islets, acts via a membrane receptor with tyrosine kinase activity and that the signal it transmits thus plays a fundamental role in the metabolism of carbohydrates, lipids and proteins. Its deficiency, due to the autoimmune destruction of the β cells in the course of so-called “type I” diabetes, is rapidly lethal following profound metabolic disorders. The three major target tissues for insulin are the liver, adipose tissue and muscle.

Insulin receptors are present on all types of cell in the body, and the effect of insulin on these cells in culture, other than the three types mentioned above, is accepted as being able to induce DNA synthesis, translation of messenger RNAs and assimilation of glucose. However, a very similar receptor, that of the insulin-like growth factors (IGFS), is co-expressed in most cells. Each of the ligands can also bind the receptor of the other ligand, but the possible functional significance of this is unknown. It nevertheless remains that the absence of insulin in children and adults has the disastrous effects mentioned above, essentially caused by liver failure.

At the present time, no example is known, either in man or animals, of a mutation that abolishes the synthesis of insulin (null mutation) and, consequently, the possible role played by insulin in the embryo during its development is unknown. On the basis of experiments with cells in culture, insulin is considered as being an important factor in the growth and differentiation of nerve cells. Maternal insulin does not cross the foeto-placental barrier. However, it has been known for several years that one of the two insulin genes, that of insulin 2, is transcribed in the mouse embryo in the primitive structures of the central nervous system. However, the role of insulin in the development of the central nervous system is unknown.

Moreover, foetal growth is attributed to the IGFs and no role of insulin is acknowledged at the present time.

One aspect of the invention is to provide an experimental model for screening medicinal products which are active on pathologies involving insulin.

One aspect of the invention is to provide transgenic mammals in which at least one of the genes coding for insulin is no longer expressed.

One aspect of the invention is to provide transgenic mammals in which the gene coding for insulin 1 is no longer expressed.

One aspect of the invention is to provide transgenic mammals in which the gene coding for insulin 2 is no longer expressed.

One aspect of the invention is to have available the restriction map for the insulin genes, thus making it possible to reclone fragments of insulin genes and of their flanking regions originating from the mouse line 129, given that the embryonic strain (ES) cells originate from the same mouse line 129.

The invention relates to the use of a non-human transgenic mammal in which at least one of the alleles for at least one of the two genes coding for the endogenous insulin is rendered functionally inoperative with respect to the expression of insulin, for the determination of medicinal products which are active on pathologies involving insulin.

Observation of the mutant mice obtained has made it possible to show for the first time that insulin is not essential to the development of the structures in the central nervous system.

Observation of the mutant mice obtained has made it possible to establish for the first time that insulin is involved in the process of foetal growth, since these mice show a retardation of foetal growth of greater than 20%.

According to one advantageous embodiment, the invention relates to the use of a non-human transgenic mammal in which the expression of endogenous insulin 1 and/or of endogenous insulin 2 is suppressed relative to normal expression, in particular in the cells of the pancreas.

In the text hereinbelow, the term “Ins1” denotes the insulin 1 gene and the term “Ins2” denotes the insulin 2 gene.

The term “mammal” includes all mammals expect for humans, advantageously rodents and in particular mice.

The term “transgenic animal” denotes an animal into whose genome has been introduced an exogenous gene construct, which has been inserted either randomly into a chromosome, or very specifically at the locus of an endogenous gene, resulting, in this latter case, in the impossibility of expressing this endogenous gene since it is either interrupted or entirely or partially replaced by a construct such that it no longer allows expression of the endogenous gene, or alternatively a construct which, in addition to the deletion of the endogenous gene, introduces an exogenous gene. Such animals will be referred to as “knock-out” animals or animals in which the abovementioned endogenous gene is invalidated.

Normal expression of the insulin gene can be defined in the following way:

1) Determination of the messenger RNA corresponding to one of the genes coding for insulin. This is possible by retrotranscription of the messenger RNA starting with an identical primer for the two messengers insulin 1 and insulin 2, followed by amplification according to a geometrical progression of the complementary DNAs generated, by the action of a polymerase (reverse transcriptase-polymerase chain amplification: RT-PCR), the two homologous, amplified fragments then being separated by electrophoresis, by means of a restriction polymorphism MspI which makes it possible to generate a 71 base-pair fragment for insulin 1 and a 112 base-pair fragment for insulin 2. The common primers used for the RT-PCR are 5′-GGCTTCTTTCTACACACCCA-3′ and 5′-CAGTAGTTCTCCAGCTGGTA-3′, and the probe common to the two products is an oligonucleotide 5′-ACAATGCCACGCTTCTG-3′.

2) Determination of the amount of protein corresponding to one of the genes coding for insulin. This is possible using antibodies which recognize the product of each gene, or alternatively by electrophoretic separation of the two insulins each revealed by a common, commercially available anti-insulin antibody.

The term “functionally inoperative” means that, relative to normal expression defined above, there is no insulin expression and the corresponding messenger RNA is totally absent.

The absence of expression of the gene for insulin 1 and/or 2 can be determined in the following way:

1) Mice in which the gene coding for insulin has been replaced with a construct such that it can no longer be expressed are first characterized relative to DNA by analysis using the DNA transfer method (Southern blot), with the aid of a probe consisting of a fragment containing all or part of the region of the gene coding for insulin, this probe being defined in the examples.

The hybridization of this probe clearly shows that the DNA band corresponding to one of the wild-type insulins (i.e. insulin types normally present in the animals) is no longer present in the homozygous animals with respect to the mutation, but instead is replaced with a band corresponding to the modified genome.

2) Analysis by RT-PCR of the RNAs extracted from tissues expressing at least one of the insulins, in particular pancreatic tissue, shows that there is no longer any RNA expression corresponding to the wild-type gene. The primers and the probe used have been defined above in the paragraph entitled “Determination of the messenger RNA corresponding to one of the genes coding for insulin”.

The cells in which the expression of a gene coding for one of the insulins is suppressed are essentially pancreatic cells.

In the context of the invention, suppression of the expression of the insulin 2 gene also occurs in the other tisues in which it is expressed, in particular the vitelline sac, the brain and the thymus.

The invention relates more particularly to a non-human transgenic mammal, or mammalian cells, in which at least one of the alleles of at least one of the two genes coding for the endogenous insulin is rendered functionally inoperative with respect to the expression of insulin.

According to one specific embodiment, the animals of the invention are such that one of the two alleles of the gene coding for insulin 1 is rendered functionally inoperative with respect to the expression of insulin 1.

According to another embodiment, the animals of the invention are such that the two alleles of the gene for insulin 1 are rendered functionally inoperative with respect to the expression of insulin 1. In other words, there is no longer any expression of insulin 1.

In this case, the over-expression of the gene for insulin 2 partially counterbalances the absence of expression of insulin 1; the animals survive and reproduce without, however, any apparent pathology.

According to one specific embodiment, the animals of the invention are such that one of the two alleles of the gene coding for insulin 2 is rendered functionally inoperative with respect to the expression of insulin 2.

According to another embodiment, the animals of the invention are such that the two alleles of the gene for insulin 2 are rendered functionally inoperative with respect to the expression of insulin 2. In other words, there is no longer any expression of insulin 2.

In this case, over-expression of the gene for insulin 1 counterbalances the absence of expression of the gene for insulin 2; the animals can have a transient sugar diabetes at birth, which disappears after a few days, but they survive and reproduce without any apparent pathology.

According to another embodiment, the animals are such that one of the two alleles of the gene coding for insulin 1 is rendered functionally inoperative with respect to the expression of insulin 1 and the two alleles of the gene for insulin 2 are rendered functionally inoperative with respect to the expression of insulin 2.

According to another embodiment, the animals are such that one of the two alleles of the gene coding for insulin 2 is rendered functionally inoperative with respect to the expression of insulin 2 and the two alleles of the gene for insulin 1 are rendered functionally inoperative with respect to the expression of insulin 1. In this case, the animals can have a transient sugar diabetes at birth, which disappears after a few days, but they survive and reproduce without any apparent pathology.

According to another embodiment, the animals of the invention are such that the two alleles of the gene for insulin 1 and of the gene for insulin 2, respectively, are rendered functionally inoperative. In this case, there is no production of insulin. The animals have a sugar diabetes with acidoketosis which develops from the start of feeding and is lethal within 2 to 3 days. This diabetes is sensitive to the administration of insulin. These animals are denoted by the term “double-nullizygous”.

The invention relates in particular to a non-human mammal or mammalian cells in which at least one of the alleles coding for insulin 1 and/or insulin 2 is replaced with a gene capable of coding for a protein which has enzymatic activity.

As genes capable of coding for a protein which has enzymatic activity, mention may be made of β-galactosidase, luciferase, chloramphenicol acetyltransferase, and an aminoglycosyl phosphotransferase.

The gene with enzymatic activity can be either under the control of the insulin gene promoter or under the control of its own promoter.

The gene capable of coding for an enzymatic protein preferably replaces at least one of the alleles of the gene for insulin 2.

According to another embodiment of the invention, the gene capable of coding for an enzymatic protein replaces at least one of the alleles of the gene for insulin 1.

According to one advantageous embodiment, the invention relates to a non-human transgenic mammal, or mammalian cells, in which expression of the endogenous gene coding for insulin 1 and that of the endogenous gene coding for insulin 2 are suppressed, and which contains a transgene for expressing human insulin.

The invention also relates to the use of a transgenic mammal as defined in the above paragraph.

The transgene for expressing exogenous human insulin correspond to a human DNA fragment containing the unit for transcription of the insulin gene and its flanking regions, in particular an 11 kilobase fragment.

According to one advantageous embodiment, the invention relates to a non-human transgenic mammal, or mammalian cells, in particular βcells, in which expression of the endogenous gene coding for insulin 1 and that of the endogenous gene coding for insulin 2 are suppressed, and which, in addition, contains a transgene for expressing human insulin. In this case, the insulin produced and stored in the β cells of the pancreas of these animals is exclusively human insulin; the animals are not diabetic, they survive and reproduce without any apparent pathology and genetically stable lines of these animals exist.

According to one advantageous embodiment, the invention relates to a non-human transgenic mammal, or mammalian cells, in particular β cells, in which expression of the endogenous gene coding, for insulin 1 and that of the endogenous gene coding for insulin 2 are suppressed, and which, in addition, contains a transgene comprising the sequence coding for the T antigen of the SV40 virus under the control of the rat insulin 2 gene promoter, and which allows induction of the proliferation of the β cells.

According to one advantageous embodiment, the invention relates to a non-human transgenic mammal, or mammalian cells, in particular β cells, in which expression of the endogenous gene coding for insulin 1 and that of the endogenous gene coding for insulin 2 are suppressed, and which, in addition, contains, on the one hand, a transgene for expressing human insulin, and, on the other hand, a transgene comprising the sequence coding for the T antigen of the SV40 virus under the control of the rat insulin 2 gene promoter which has been premodified so as to induce termination of its transcription under the effect of the presence of tetracycline, and allowing stoppage of the proliferation of the β cells in the presence of tetracycline.

According to one advantageous embodiment, the invention relates to a non-human transgenic mammal, or mammalian cells, in particular β cells, in which expression of the endogenous gene coding for insulin 1 and that of the endogenous gene coding for insulin 2 are suppressed, and which also contains, on the one hand, a transgene for expressing human insulin, and, on the other hand, a construct containing the transgene comprising the sequence coding for the T antigen of the SV40 virus under the control of the rat insulin 2 gene promoter which has been premodified so as to induce its transcription under the effect of the presence of a substance, in particular an antibiotic, a hormone, a cytokine or a growth factor, and which allows induction of the proliferation of the β cells in the presence of the abovementioned substance.

The invention also relates to cells encapsulated in an inert material capable of grafting these cells in vivo under conditions sheltered from the immune system.

The term “inert material” denotes a physicochemical complex or substance which does not modify the grafted biological material and which does not induce an immunological reaction in the host, thus making this material capable of grafting.

The term “conditions sheltered from the immune system” means that the live grafted cells are separated from the host's immune system such that the cells of the immune system, the antibodies and other rejection factors, cannot attack them.

The invention also relates to a non-human mammal, or mammalian cells, resulting from the crossing of the animals defined above, or from crossing of any one of the animals defined above, with another animal of the same species.

The invention also relates to cells cultured using the non-human transgenic mammals described above.

According to one advantageous embodiment, the invention relates to cell cultures containing one of the abovementioned transgenic constructs.

These cell cultures can be obtained either using cells taken from transgenic animals as defined above or from cell lines using the abovementioned transgenic constructs, it being possible for this second possibility to be carried out using standard cell transfection techniques.

The invention also relates to a non-human transgenic mammal as obtained by introduction into a blastocyte of embryonic strain cells (ES cells) whose genome comprises one of the abovementioned transgenic constructs, obtained by homologous recombination, selection of chimeric animals according to a criterion corresponding to the ES line, crossing of the selected animals with mice, in particular C57 Black 6 mice, in order to obtain animals which are heterozygous relative to one of the abovementioned constructs and, optionally, crossing of two heterozygotes in order to obtain an animal which is homozygous relative to one of the abovementioned constructs.

The homozygote has a 50/50 129/C57 Black 6 genetic background. It is possible to return to a C57 Black 6 genetic background by homozygous crossing with C57 Black 6 mice over at least 12 generations.

C57 Black 6 mice are advantageously chosen, since this genetic background is more favourable for certain behavioural experiments.

The invention also relates to a transgenic mammal as produced by crossing transgenic animals which express one of the transgenic constructs defined above.

The invention also relates to the process for obtaining a transgenic model for studying pathologies involving insulin and for treatment of these pathologies, comprising:

replacement of all or part of the gene coding for insulin 1 with the neo gene, and in particular replacement of the nucleotide fragment ranging from position −0.8 kilobase to position +0.687 kilobase (with reference to the transcript origin located at position +1), and in particular

replacement of at least one of the alleles for the endogenous gene coding for insulin 1 , in cells, in particular mouse embryonic strain (ES) cells, with a construct as obtained in the following manner and denoted by pIns1^{neo},

sub-cloning of a 7.2 kb ApaI/XhoI fragment (corresponding to the flanking region downstream (at the 3′ end) of Ins1) into a plasmid pSK⁺ predigested with ApaI and XhoI, resulting in p4,

sub-cloning of another fragment BamHI/HindIII (corresponding to the region at the 5′ end of Ins1) into pSK⁺, giving pR1,

the vector used to manufacture the recombination vector being derived from pSK⁺, containing the neo gene (BamHI fragment originating from pMC1 POLA -Stratagene) at the BamHI site, and the tk gene (XhoI/HindIII fragment originating from pMCtk described in Liu et al., Cell, 1993, Vol. 75, 59-72) between the XhoI and HindIII sites and denoted by pNTK,

cloning of the NotI/PvuII fragment of pR1 corresponding to the fragment of homologous sequence at the 5″ end into pNTK between the NotI and XbaI sites, giving pR2, in which the 6.5 kb HindIII fragment of p4 (corresponding to the fragment of homologous sequence at the 3′ end) has been cloned into the HindIII site giving pIns1^{neo},

and/or replacement of all or part of the sequence coding for the insulin 2 gene with a sequence coding for a protein with enzymatic activity, and in particular replacement of the nucleotide fragment ranging from position +21 to position +856 base pairs (with reference to the transcript origin located at position +1), and, in particular, replacement of the coding sequence of one or two alleles of the endogenous gene coding for insulin 2 with that of the LacZ gene of Escherichia coli in mouse embryonic strain (ES) cells, and in particular with a construct as obtained in the following way and denoted by pIns2^{Zneo}

sub-cloning of a 7 kb EcoRI fragment, corresponding to the flanking region at the 3′ end of the insulin 2 gene, into the EcoRI site of pSK⁺, giving p12,

destruction of an NsiI site present in the 7 kb insert of p12, giving p12ΔNsiI,

sub-cloning of a 5 kb XbaI fragment containing the insulin 2 gene also into pSK⁺, giving p13,

synthesis of the −950/+20 region of the insulin 2 gene by a polymerase chain reaction (PCR) using the following nucleotide primers: 5′-CGCTCTAGACCCTCCTCTTGCATTTCAAA-3′ and 5′-CGCATGCATGTAGCGGATCACTTAGGGT-3′ these primers bringing XbaI and NsiI sites into the PCR product obtained,

cloning of the PCR product upstream of the sequence coding for the LacZ gene into the pGN vector (Le Mouellic et al., 1990, PNAS, 87, 4712-4716) between the XbaI and NsiI sites,

destruction of the NsiI site, giving p15,

replacement of the XbaI/SfiI fragment of p15 with an XbaI/SfiI fragment originating from p13, giving p16,

modification of the pNTK vector as defined above by inserting an NsiI linker therein, into the HindIII site, giving p10,

cloning of the XbaI/XhoI fragment originating from p16 (containing both the 2.7 kb fragment of homologous sequence at the 5′ end and LacZ) into p10 between the XbaI and SpeI sites, giving p17,

cloning of the 5.5 kb SmaI/EcoRI fragment originating from p12ΔNsiI (corresponding to fragment of homologous sequence at the 3′ end) into the NsiI site of p17 using NsiI linkers and giving pIns^{Zneo},

introduction of the abovementioned cells into embryos, in particular non-human mammalian blastocytes, in particular mouse blastocytes,

selection of male chimeric animals according to a criterion corresponding to the ES line,

crossing of the selected animals with mice, in particular C57BL/6 mice, giving animals which are heterozygous relative to one of the constructs as defined above, and

optionally, crossing of two heterozygotes in order to obtain an animal which is homozygous relative to one of the constructs as defined above,

optionally, crossing of homozygotes relative to each of the constructs defined above in order to obtain double-heterozygotes for each of the constructs,

optionally, crossing of the double-heterozygotes, giving, in particular, double-homozygous animals.

The invention also relates to a process for screening medicinal products which are active on pathologies involving insulin, in particular diabetes, comprising the administration of a medicinal product to be tested to a transgenic non-human mammal or to transgenic non-human mammalian cells

containing, in the place of at least one of the alleles of the endogenous gene coding for insulin 2 , a sequence coding for a protein with enzymatic activity, and in particular the nucleotide fragment ranging from position +21 base pairs to position +856 base pairs, and in particular a construct pIns2^{Zneo} as defined above, and

optionally containing, in the place of at least one of the alleles of the endogenous gene coding for insulin 1, the neo gene and in particular the nucleotide fragment ranging from position −0.8 kilobase to position +0.856 kilobase, and in particular a construct pIns1^{neo} as defined above,

determination of the β-galactosidase activity of the β cells.

The invention also relates to a transgenic construct in which:

all or part of the sequence of an allele of the endogenous gene coding for insulin 1 is replaced with the neo gene, and in particular the nucleotide fragment ranging from position −0.8 kilobase to position +0.687 kilobase, and in particular

at least one of the alleles of the endogenous gene coding for insulin 1 is replaced in cells, in particular mouse embryonic strain (ES) cells, with a construct as obtained in the following manner and denoted by: pIns1^{neo},

sub-clonig of a 7.2 kb ApaI/XhoI fragment (corresponding to the flanking region downstream (at the 3′ end) of Ins1) into a plasmid pSK⁺ which has been predigested with ApaI and XhoI, resulting in p4,

sub-cloning of another BamHI/HindIII fragment (corresponding to the region at the 5′ end of Ins1) into pSK⁺, giving pR1,

the vector used to manufacture the recombination vector being derived from pKS⁺, containing the neo gene (BamHI fragment originating from pMC1 POLA -Stratagene) at the BamHI site, and the tk gene (XhoI/HindIII fragment originating from pMCtk described in Liu et al., Cell, 1993, Vol. 75, 59-72) between the XhoI and HindIII sites and denoted by PNTK,

clonig of the NotI/PvuII fragment of pR1 corresponding to the fragment of homologous sequence at the 5′ end into pNTK between the NotI and XbaI sites, giving pR2, in which the 6.5 kb HindIII fragment of p4, corresponding to the fragment of homologous sequence at the 3′ end, has been cloned into the HindIII site, giving pIns1^{neo},

and/or in which all or part of the sequence of an allele of the endogenous gene coding for insulin 2 is replaced with a sequence coding for a protein with enzymatic activity, and in particular the nucleotide fragment ranging from position +21 to position +856, and in particular,

the coding sequence of one or two alleles of the endogenous gene coding for insulin 2 is replaced with that of the LacZ gene of Escherichia coli in mouse embryonic strain (ES) cells, and in particular with a construct as obtained in the following manner and denoted by pIns2^{Zneo},

sub-cloning of a 7 kb EcoRI fragment, corresponding to the flanking region at the 3′ end of the insulin 2 gene, into the EcoRI site of pSK⁺, giving p12,

destruction of an NsiI site present in the 7 kb insert of p12, giving p12ΔNsiI,

sub-cloning of a 5 kb XbaI fragment containing the insulin 2 gene also into pSK⁺, giving p13,

synthesis of the −950/+20 region of the insulin 2 gene by a polymerase chain reaction (PCR) using the following nucleotide primers: 5′-CGCTCTAGACCCTCCTCTTGCATTTCAAA-3′ and 5′-CGCATGCATGTAGCGGATCACTTAGGGT-3′ these primers bringing XbaI and NsiI sites into the PCR product obtained,

cloning of the PCR product upstream of the sequence coding for the LacZ gene into the pGN vector (Le Mouellic et al., 1990, PNAS, 87, 4712-4716) between the XbaI and NsiI sites,

destruction of the NsiI site, giving p15,

replacement of the XbaI/SfiI fragment of p15 with an XbaI/SfiI fragment originating from p13, giving p16,

modification of the PNTK vector as defined above by inserting an NsiI linker therein, into the HindIII site, giving p10,

cloning of the XbaI/XhoI fragment originating from p16 (containing both the 2.7 kb fragment of homologous sequence at the 5′ end and LacZ) into p10 between the XbaI and SpeI sites, giving p17,

cloning of the 5.5 kb SmaI/EcoRI fragment originating from p12ΔNsiI (corresponding to fragment of homologous sequence at the 3′ end) into the NsiI site of p17 using NsiI linkers and giving pIns2^{Zneo}.

The invention also relates to the genomic DNA of insulin 1 of the consanguine mouse line 129, characterized by the following restriction sites, with reference to the transcript origin located at position +1:

upstream of the site +1:

two PvuII sites (at −8.4 and −0.8 kilobases) two BamIII sites (at −3.9 and 3.3 kilobases) one HindIII site (at −8.3 kilobases) one ApaI site (at −0.4 kilobases)

downstream of the site +1:

one SmaI site (at +388 base pairs)

two PvuII sites (at +479 base pairs and +8 kilobases)

two HindIII sites (at +687 base pairs and +7.3 kilobases)

one XhoI site (at +7.8 kilobases).

The invention also relates to the genomic DNA of insulin 2 of the consanguine mouse line 129, characterized by the following restriction sites, with reference to the transcript origin located at position +1:

upstream of the site +1:

one NsiI site (at −10.8 kilobases)

two EcoRI sites (at −5.4 kilobases and −455 base pairs)

one XbaI site (at −2.7 kilobases)

one SfiI site (at −239 base pairs)

downstream of the site +1:

two EcoRI sites (at +378 base pairs and 7.9 kilobases)

one SmaI site (at +856 base pairs)

one XbaI site (at +2.2 kilobases)

one NsiI site (at +4.2 kilobases)

one XhoI site (at +7 kilobases).

Conclusions

The advantage of the mutant mice of the invention in the study of diabetes, compared with already-existing animal models, such as mice of the NOD line or rats of the BB line, is that the absence of insulin is total from birth, this absence exists from the embryonic stage onwards and it exists without the autoimmune disease, which is the cause of the disease in the abovementioned animals and of type I human diabetes. They make it possible to explore the actual role of the absence of insulin without an associated immnune component.

Their advantage relative to the experimental induction of a diabetes by destruction of the pancreatic β cells, which is achieved using drugs such as alloxan or streptozotocin, is that the β cells are unharmed in the total absence of insulin, which excludes the interference of the cytolytic effects in the syndrome of absence of insulin and makes it possible to study the effect of the absence of insulin in the presence of all the cellular factors of the body.

The mutant mice in which only one, two or three insulin genes are rendered invalid show no detectable chronic diabetic syndrome. The mutant mice of the invention make it possible to determine whether or not, at an advanced age, these animals develop vascular complications, which are common in diabetics, even diabetics who appear to be suitably equilibrated by the insulin therapy. These complications are responsible for blindness, renal insufficiency and arteritis. It is possible that the mice defined above are poorly equilibrated in a hitherto imperceptible manner, but that they can represent an animal model for vascular complications of diabetes, this model not existing at all at the present time.

Mice are currently the only animals for which it is known how to derive pancreatic β cell lines reproducibly. Furthermore, these cells conserve their essential functional properties: synthesis and storage of insulin, and secretion induced by glucose. The possiblity of deriving such cells from mutant animals devoid of insulin synthesis is an important opening in the perspective of developing β cells which can be used in a cellular treatment of diabetes. The reason for this is that the introduction of a human insulin transgene into mutant mice or even directly into the β cells already established in culture makes it possible to obtain β cells which can optionally be grafted into man (by means of cellular encapsulation processes, allowing the grafted cells to be protected from the host's immune system) which, under the physiological control of the host, secrete human insulin, i.e. an intrinsic protein which does not itself induce an immune reaction.

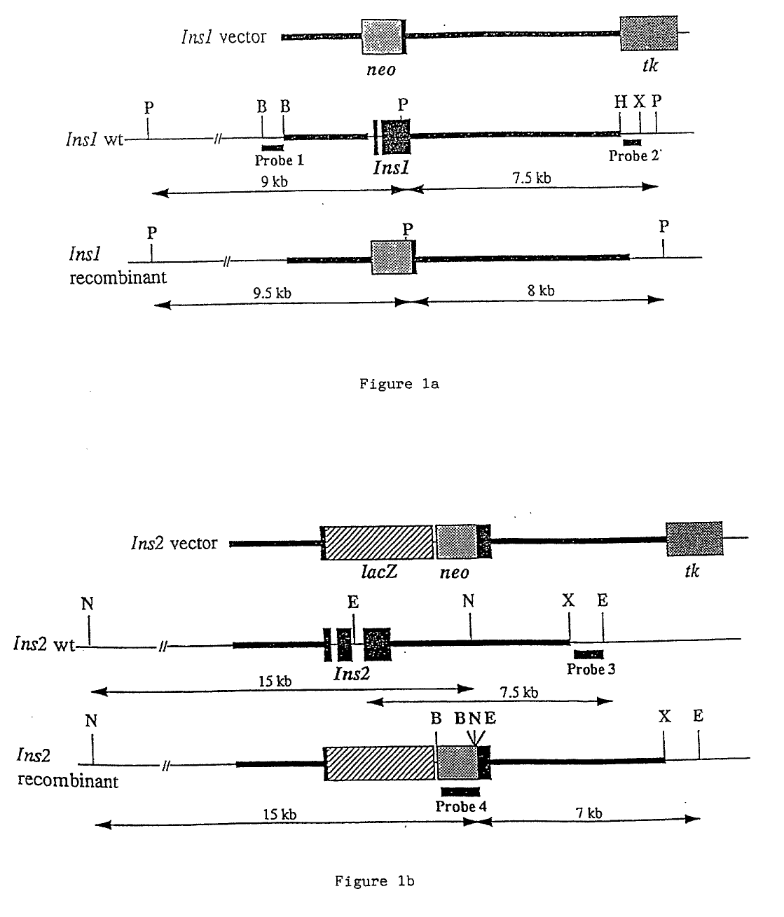

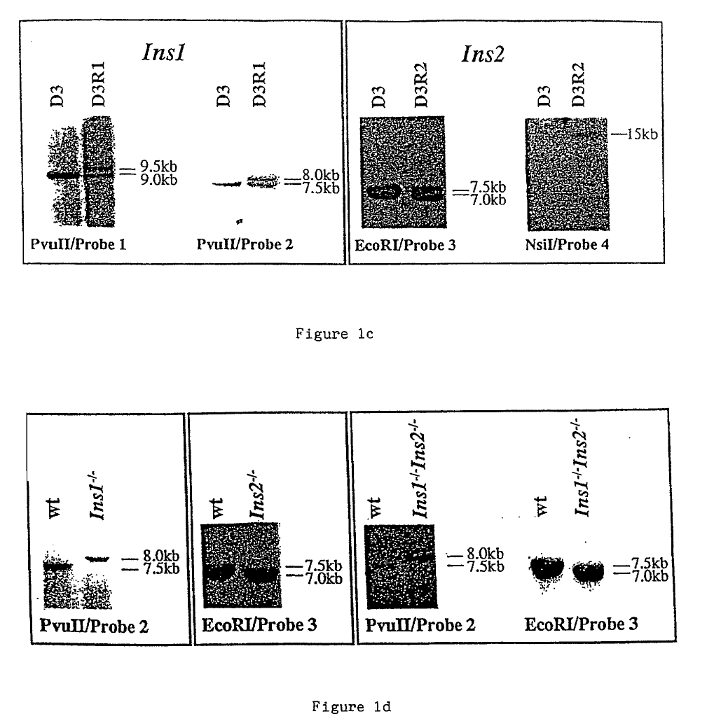

FIG. 1: This represents the targeted interruption of Ins1 (FIG. 1a) and of Ins2 (FIG. 1b).

The structures of the targeting vectors, as well as the wild-type (wt) and the recombinant alleles, are represented, respectively, in FIG. 1a for Ins1 and in FIG. 1b for Ins2. The restriction enzymes and the probes used for the genotyping of the mouse and cellular DNA are indicated. The autoradiograms of the Southern blot analyses confirm the production of the embryonic strain (ES) cells (D3) bearing a recombinant allele for Ins1 (D3R1) or Ins2 (D3R2) in (c) and of Ins1⁻/⁻ and of Ins2⁻/⁻ and of the mutant mice which are double-homozygotes in (d) neo=neomycin phosphotransferase gene; tk=gene for the thymidine kinase of the type 1 herpes simplex virus, B=BamHI; E, EcoRI; H, HindIII; N, NsiI; P, PvuII; X, XbaI.

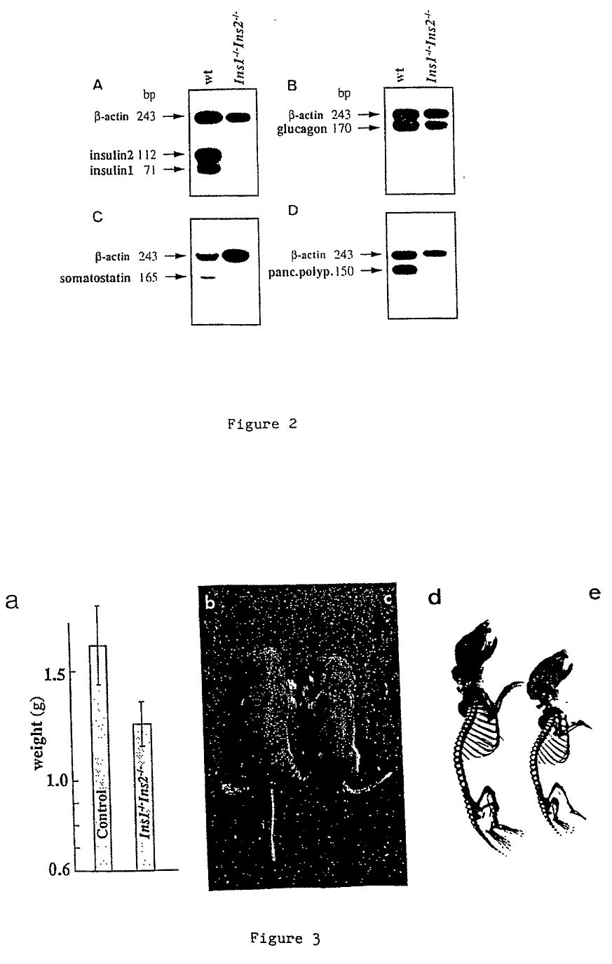

FIG. 2: RT-PCR analysis of the expression of Ins1l/Ins2 in the pancreas of wild-type mice and double-nullizygous mutant mice. The mRNA of β-actin is amplified as control.

FIG. 3: Retardation of growth (a) of newborn double-nullizygous mice (n=11) compared with control animals.

The morphology of the control (b) and of an insulin-deficient young mouse (c). The skeleton of an insulin-deficient newborn mouse (e) is smaller than that of the control (d).

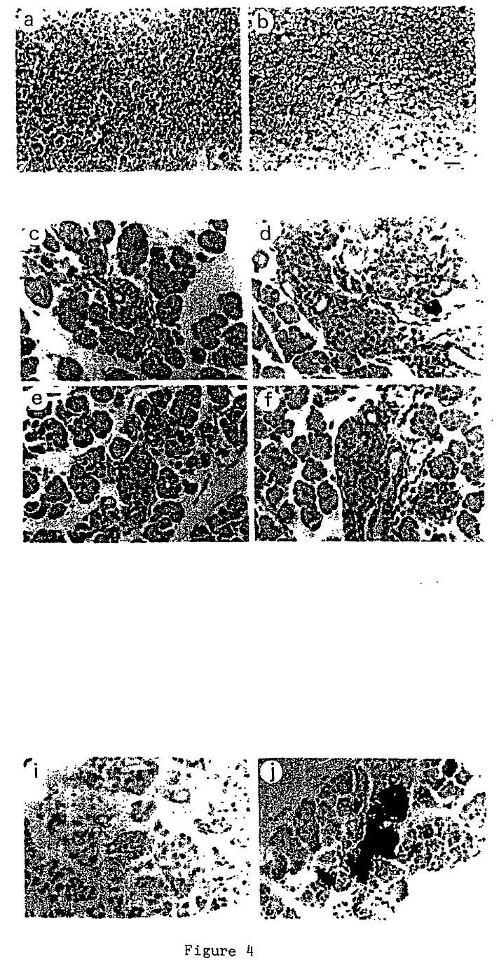

FIG. 4: Analysis of the liver and pancreas of wild-type mice and double-nullizygous mice.

Histological analysis of liver sections from wild-type mice (a) and insulin-deficient mice (b).

Immunocytochemical staining of pancreatic sections from wild-type mice (c, e) and insulin-deficient mice (d, f) using antibodies raised against peptide 1-C (c, d), peptide 2-C (e, f).

The expression of LacZ in the pancreatic sections from insulin-deficient mice is visualized by staining with X-Gal (j). The background staining obtained with wild-type animals is represented in (i) Scale: a, b: 10μ; c, j=8μ.

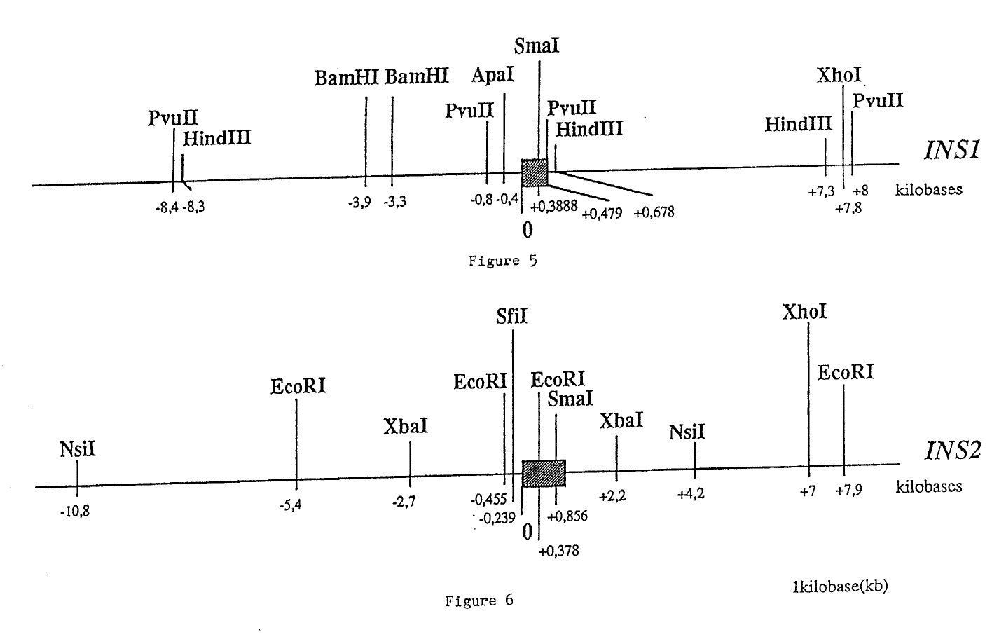

FIG. 5: Restriction map of the insulin 1 gene of the consanguine mouse line 129.

FIG. 6: Restriction map of the insulin 2 gene of the consanguine mouse line 129.

### EXAMPLE 1

### Materials and Methods

The null mutation of the insulin 1 gene (Ins1) or that of the insulin 2 gene (Ins2) required the following steps:

1) cloning of the gene into a DNA library of consanguine mice of the line 129,

2) establishment of the restriction map of the cloned locus,

3) construction of the recombination plasmid vector,

4) electroporation of this vector into embryonic strain cells (ES cells) of male mice of the line 129,

5) selection of ES cells which have incorporated the recombination vector and molecular characterization of the mutated locus,

6) micro-injection of ES cells bearing the mutation into mouse blastocytes, transfer of these blastocytes into the oviduct of carrier females and production of male chimeric mice,

7) crossing of the chimers with wild-type mice and identification, in the first-generation descendants, of individuals derived from the ES cell and bearing the null mutation (in the heterozygous state),

8) self-crossing of heterozygous mice and identification, in the descendants, of mice bearing the null mutation in the homozygous state (nullizygous mice),

9) maintenance and propagation of the line by self-crossing of nullizygotes.

The mice bearing a null mutation of the two alleles of the gene Ins1 and of the gene Ins2 (double-nullizygotes) are obtained in two steps. Firstly, crossing of a mouse which is nullizygous for Ins1 with a mouse which is nullizygous for Ins2 produces a first generation of mice which are all heterozygous for each of the mutations. Next, self-crossing of these mice generates double-nullizygous mice in a frequency of 0.0625 (i.e. on average one mouse out of 16).

Cloning of the gene into a DNA library of consanguine mice of the line 129

Complementary DNAs (cDNAs) corresponding, on the one hand, to Ins1, and, on the other hand, to Ins2, labelled with ³²Phosphorus, were used as probes for screening the library.

A commercially available mouse 129 library (Stratagene) made it possible to clone several Ins2 λ phages. Another library, constructed at the Unité INSERM 184, made it possible to clone several other λ phages corresponding to Ins1.

The use of a variety of restriction enzymes made it possible to establish the restriction map of the various clones and to orient the various phages corresponding to one or other gene. Next, using these maps, several restriction fragments were sub-cloned.

Construction of the recombination plasmid vector

A—For Ins1, an 8.7 kilobase (kb) Hind III/SmaI fragment and an 8.2 kb ApaI/XhoI fragment (corresponding to the flanking regions upstream (at the 5′ end) and downstream (at the 3′ end) of Ins1) were separately sub-cloned into a plasmid pSK+ which was predigested with HindIII and SmaI for one, and ApaI and XhoI for the other, resulting in the plasmids p34 and p4. Another fragment BamHI/HindIII (corresponding to the 5′ region of Ins1) was also cloned into pSK+, giving pR1. The vector used to manufacture the recombination vector is derived from pKS+ containing the neo gene at the BamHI site and the tk gene between the XhoI and HindIII sites. The NotI/PvuII fragment of pR1 corresponding to the fragment of homologous sequence at the 5′ end was cloned into pJorg between the NotI and XbaI sites, giving pR2, in which the 6.5 kb HindIII fragment of p4, corresponding to the fragment of homologous sequence at the 3′ end, was cloned into the HindIII site. The result is the recombination vector for Ins1. The synthetic probes 1 and 2 (see FIG. 1) correspond, for one, to the BamHI fragment of p34, and, for the other, to the HindIII/XhoI fragment of p4.

B—For Ins2, the sequence coding for the gene was replaced with that for the LacZ gene of Escherichia coli. LacZ codes for the enzyme β-galactosidase which cleaves lactose into glucose and galactose. The use of a colourless precursor X-gal as an enzyme substrate generates an intense blue colouring product which can be visualized on anatomical or histological preparations and assayed by colorimetry. Two EcoRI fragments of 5 and 7 kb, corresponding to the flanking regions, one at the 5′ end and the other at the 3′ end of the Ins2 gene, were sub-cloned into the EcoRI site of pSK+, giving, on the one hand, p11, and, on the other hand, p12. An NsiI site present in the 7 kb insert of p12 was destroyed, giving p12ΔNsiI. A 5 kb XbaI fragment containing Ins2 was also sub-cloned into pSK+, giving p13. The region

−950/+20 of Ins2 was synthesized by a polymerase chain reaction (PCR) using the following nucleotide primers:

5′-CGCTCTAGACCCTCCTCTTGCATTTCAAA-3′ and

5′-CGCATGCATGTAGCGGATCACTTAGGGT-3′. These primers bring XbaI and NsiI sites into the PCR product obtained. This was cloned upstream of the sequence coding for the LacZ gene into the pGN vector (obtained from Philippe Brûlet) between the XbaI and NsiI sites. The NsiI site was then destroyed, giving p15. The XbaI/SfiI fragment of p15 was replaced with an XbaI/SfiI fragment originating from p13, giving p16. To construct the recombination vector, the derivative pKS+ defined above was first modified by inserting an NsiI linker therein, into the HindIII site, giving p10. The XbaI/XhoI fragment originating from p16, containing both the 2.7 kb fragment of homologous sequence at the 5′ end and LacZ, was cloned into p10 between the XbaI and SpeI sites, giving p17. The 5.5 kb SmaI/EcoRI fragment originating from p12DNsiI [sic], corresponding to the fragment of homologous sequence at the 3′ end, was cloned into the NsiI site of p17 using NsiI linkers, giving the recombination vector for Ins2. The synthetic probe 3 corresponds to the XhoI/EcoRI fragment of p12 and the probe 4 corresponds to the neo fragment (see FIG. 2).

All the manipulations of the phages, the bacteria and the DNA were carried out using the experimental protocols described in reference [1].

Generation of the mutant mice

The mouse ES cell cultures and the mouse embryo manipulations were carried out according to the methods described [2-4]. The DNAs of the recombination vectors were linearized by digestion with NotI followed by electroporation into ES cells of the D3 line. After selection by the drugs G418 (Gibco) and Ganciclovir (Syntex), 3 recombinant clones for Ins1 and 12 for Ins2 wrer identified by molecular analysis (see below). Ten to fifteen cells originating from these clones were micro-injected into the cavity of blastocytes of the mouse line C57BL/6 (B6), which were then transferred into the oviduct of pseudogestating females. Several chimeric males were obtained. They were crossed with B6 females. Two independent cell clones were used for each of the mutations.

The first-generation mice, whose coat is agouti, are derived from germinal cells derived from ES cells. The molecular analysis (see below) makes it possible to identify those which have inherited the recombinant Ins allele, i.e. the null allele.

The mice which are heterozygous for a mutation were crossed with each other. In their first-generation descendants, a quarter of the animals were found to be nullizygous, i.e. homozygous for the null mutation under consideration. The self-crossing of these nullizygous animals made it possible to establish nullizygous lines either for the Ins7 gene or for the Ins2 gene.

The crossing of a mouse which is nullizygous for Ins1 with a mouse which is nullizygous for Ins2 produces first-generation animals which are all heterozygous for each of the mutations. The self-crossing of these double-heterozygotes produces, depending on the chromosomal combinations, animals containing 0, 1, 2, 3 or 4 invalidated Ins genes. The gene combination achieved in each individual can be identified by molecular biology tests (see below).

Molecular analysis of the cells and animals

1) Analysis of the DNA restriction fragments

The presence of the null mutation in recombinant ES cells, to the exclusion of any other random insertion of the recombination vector, is detected by DNA analysis. The restriction fragments generated from the DNA are separated by electrophoresis, transferred onto a Nylon membrane on which they are incubated with a specific radioactive probe, the hybridization of which with DNA fragments of determined size, revealed by autoradiography, makes it possible to confirm the presence of the expected mutation.

For Ins1, the digestion with PvuII makes it possible to visualize, for the mutated allele, the replacement of a 9.0 kb band with a 9.5 kb band (with probe 1) and that of a 7.5 kb band with an 8.0 kb band (with probe 2). The coexistence of two 9.0/9.5 kb (probe 1) and 8.0/7.5 kb (probe 2) doublets indicates the heterozygosity of the mutation. The presence, in a mouse, of two unique bands of 9.5 kb (probe 1) and 8.0 kb (probe 2) is a sign of the homozygosity of the mutation.

For Ins2, the digestion with EcoRI makes it possible to visualize, for the mutated allele, the replacement of a 7.5 kb band with a 7.0 kb band (with probe 3), while the digestion with NsiI makes it possible to visualize the appearance of a 15 kb band (with probe 4). The existence of an EcoRI doublet with the probe 3 indicates the heterozygosity, while that of a single band of 7.0 kb indicates the homozygosity of the mutation.

2) The discrimination between the various genotypes in the intercrossings can be carried out by performing the same examination.

3) The search for the Ins1 and Ins2 transcripts is performed by a technique combining the reverse-transcription of the messenger RNA, followed by amplification of the cDNA generated according to a described protocol [5].

4) The demonstration of the precursor (pro-insulin) of each of the insulins 1 and 2 in the islets of Langerhans β cells is performed on pancreatic histological slices using a standard immunocytochemistry technique in the presence of antibodies which are specific for one or other product.

5) The mutation introduced into the Ins2 gene includes the sequence coding for the LacZ gene which is placed under the control of the regulatory sequences of the Ins2 gene. The reason for this is that the LacZ gene is now functioning like the Ins2 gene and in its place. As has already been mentioned above, the product of this gene, the enzyme β-galactosidase, is capable of cleaving a colourless precursor, generating a blue derivative. This can be seen by transparency on a whole pancreas, on histological slices. It can be demonstrated and its concentration measured by colorimetry of cellular or tissue extracts.

The transgenic mice of the invention, lacking insulin, make it possible to develop or test alternative treatment strategies for diabetes.

In particular, they make it possible to obtain β cells intended for a cellular treatment of diabetes.

1) Production of mice in which the only insulin produced is human insulin:

Transgenic mouse lines which express the gene for human insulin are available. These mice are obtained by micro-injecting into the pronucleus of zygotes (ovocyte fertilized by a spermatozoon and in which the two male and female pronuclei are always distinct) an 11 kilobase human DNA fragment containing the insulin gene (1430 base-pairs). Some of the mice derived from the development of these embryos incorporated the foreign DNA (the transgene) into one of their chromosomes. They express the transgene, have a fraction of their insulin which is human insulin and show no pathology, their overall quantity of synthesized and stored insulin being normal. They served to establish, by crossing, lines of transgenic mice for the human insulin gene. One of these lines (Tg171), in which the human transgene is incorporated into chromosome No. 18 [6] is used to introduce the human transgene into a line lacking functional mouse insulin genes.

For this, the Tg171 mice were crossed with mice bearing null alleles of Ins1 and Ins2. First-generation mice bearing a null allele for Ins1, a null allele for Ins2 and a human transgene were obtained. Their self-crossing will make it possible to obtain individuals bearing two null Ins1 alleles, two null Ins2 alleles and two alleles of the transgene. In contrast with the double-nullizygous mice, these mice survive to the neonatal period since they have a production of insulin. This insulin is exclusively human insulin.

2) Production of β cells:

Transgenic mouse lines exist for a gene construct comprising the sequence coding for the T antigen of the SV40 virus which is under the control of the rat Ins2 gene promoter (Ins-Tag transgene). These mice express the T antigen in the β cells, the effect of which is to induce their proliferation, to the same extent in vivo as when they are placed in culture. Their appreciable proliferation in culture, which does not exist for the β cells of ordinary mice, makes it possible to establish β cell lines which conserve their essential biological properties. However, it is impossible to stop their proliferation, which remains uncontrolled [7-9].

More recently, a mouse line bearing the same Ins-Tag transgene, which has been modified beforehand so as to induce termination of its transcription under the effect of the administration of tetracycline, has been constructed. The proliferation of the β cells in culture derived from such animals is stopped by the addition of tetracycline in the culture medium [10].

Other transgenic mice can be constructed, in which the Ins-Tag transgene is modified so as to induce transcription under the effect of a drug. The proliferation of the β cells, both after birth and in culture, is possible only after administration of this drug and ceases with it. This case is operationally much more advantageous for envisaging a cell therapy.

Introduction of the transgene coding for the T antigen, in one of the three forms described above, into the genetic inheritance of mice which produce only human insulin, makes it possible to establish different β cell lines which can be transplanted into a host.

Cells encapsulated in an inert material have been used for a number of years to graft these cells in vivo under conditions in which they are sheltered from the immune system. Micro-encapsulation, in sodium alginate microspheres, of islets of Langerhans, followed by grafting them into the diabetic animal, makes it possible to eliminate the hyperglycaemia of these animals [11].

The use of mouse β cells which produce only human insulin in such a protocol makes it possible to test the efficacy of this cellular treatment of mouse or rat diabetes. Such cells can be used in clinical trials, especially if their in vivo proliferation is dependent on the administration of a drug.

Another cell therapy route is represented by the attempts to modify fibroblasts, or other cell types other than β cells, such that they secrete insulin in a manner regulated by glucose. The mutant mice lacking insulin can be used to test the efficacy of these treatments.

3) Test of genic therapy strategies for treating diabetes:

3.1) Production of a transgenic insulin in the liver or in any other location

A variety of genetic manipulations resulting in the expression and secretion of insulin or of an analogue in a tissue other than the pancreatic β cells can open an alternative route to the treatment of diabetes. The efficacy of the regulation of the homeostasis of glucose and the sustained maintenance of the secretion can be tested in mutant mice lacking any insulin of pancreatic origin.

3.2) Constitutive expression of hepatic glucokinase

Many metabolic complications of sugar diabetes depend essentially on hepatic disorders of glucose metabolism. It is generally considered that many glycolytic enzymes are induced by the insulin signal. However, it is possible that this is true only for the first enzyme in the metabolic chain, glucokinase, which is required for the conversion of glucose into glucose-6-phosphate, and that the other enzymes are in fact only induced by the diabetic hyperglycaemia. In this case, the introduction into mice, by transgenesis, of a mutated glucokinase gene, in order to make this enzyme constitutively active, makes it possible to correct metabolic diabetic disorders substantially.

4)Identification (screening) of drugs which can modify the expression of the insulin gene:

Mice bearing a mutation of an Ins2 gene have the LacZ gene which is induced and active, as is the Ins2 gene. Introduction of the transgene coding for the T antigen Ins-Tag in these mice, by crossing suitable mice, makes it possible to develop, using these animals, one or more β cell lines whose β-galactosidase activity serves as an indicator for screening drugs of any nature which are capable of inducing or, on the other hand, of suppressing the transcription of the Ins2 gene.

5) Evaluation of insulin analogues for treatment:

In more general terms, mice which are double-nullizygous for Ins and which express the human insulin gene can be used to assess the possible immunogenic effect of synthetic insulins or of recombinant insulins which can be used in man for the treatment of diabetes or in other indications.

6) Study of diabetic complications:

Mice which are nullizygous for the Ins2 gene have no obvious glycaemic disorders. However, at birth, some of them exhibit transient glycosuria for a few hours. This observation is explained by the fact that, before birth, the level of transcripts of the other gene, Ins1, is similar to that of the non-mutant mice, but that, from birth onwards, there is a compensation for the absence of Ins2 by means of the increased transcriptional activity of Ins1. However, this massive transcriptional compensation has not been found for the Ins2 gene as regards mice which are nullizygous for Ins1.

The transient diabetes observed in newborn mice which are nullizygous for Ins2 has been observed, in a more sustained manner (few days), in some of the animals which have three invalidated copies of Ins genes.

These mutant animals are examined in order to detect whether or not they carry vascular anomalies. At the present time, no animal model exists for vascular complications of sugar diabetes, and the mutants which are the subject of this invention may be a first example thereof.

7) Study of the role of insulin in autoimmunity:

In the normal state, the Ins2 gene, and not the Ins1 gene, is transcribed in the thymus of young mice. The same phenomenon has been described for the single insulin gene in man. The availability of mice which are nullizygous for Ins2 offers the opportunity of examining the role of the expression of Ins2 in the thymus under the normal establishment of autoimmunity. Analysis of this question requires the use of specific T hybridomas established in other mouse lines and which can be used only in these lines. Introduction of the nullizygosity for Ins2 in one of these lines will be carried out by suitable crossings. Thus, it is possible to develop a line which is nullizygous for Ins2 and which can be analysed with the available hybridomas.

The role of insulin as an auto-antigen in the development of the auto-immunized diabetes known as juvenile diabetes or type I diabetes can be explored using double-nullizygous NOD mice, obtained by suitable crossings. If it is found that these mice no longer develop autoimmune diabetes, the exploration is conducted by introducing insulin transgenes bearing mutations in the epitopes suspected of being in doubt in the autoimmune disease.

### EXAMPLE 2

Production of mice in which all the insulin produced is human insulin.

These mice were obtained by introducing a human insulin transgene into a mouse line whose Ins1 and Ins2 genes have been rendered invalid by homologous recombination.

The human transgene is an 11 kilobase fragment of genomic DNA comprising the transcription unit of the human (pro)insulin gene (Insh), surrounded by an upstream DNA fragment (5′ fragment) of about 4 kilobases and a downstream fragment (3′ fragment) of about 5.5 kilobases.

It was introduced into the genetic inheritance of a mouse line by micro-injection of this purified fragment into a pronucleus of a second-generation mouse zygote from a crossing between the two consanguine lines C57B1/6 and CBA/He.

The transgenic line was identified by genomic DNA analysis by the Southern blot method. The functional activity of the Insh transgene was characterized by the demonstration and assay of human peptide C in the urine by means of a radioimmunological test (RIA), the demonstration of a human insulin transcript in the pancreatic total RNA preparations and the demonstration of the initiation of this transcript at the physiological site, the demonstration that the human protein produced is found only in the β cells of the endocrine pancreas by immunocytofluorescence using specific antibodies, and the demonstration that it is co-localized with the mouse insulins in the same secretion granules of these β cells.

The transgene was located on the mouse chromosome No. 18.

The quantification established that the human insulin represents 47% of the total insulin synthesized and stored in the transgenic islets. Reference may be made to the following references:

Bucchini D., Ripoche M- A., Stinnakre M- G., Desbois P., Lorès P., Monthioux E., Absil J., Lepesant J- A., Pictet R., Jami J. (1986)—“Pancreatic expression of human insulin gene in transgenic mice”; Proc. Natl Acad. Sci., USA 83: 2511-2515.

Michalova K., Bucchini D., Ripoche M- A., Pictet R., Jami J. (1988)—“Chromosome localization of the human insulin gene in transgenic mouse lines”; Human Genet. 80: 247-252.

Bucchini D., Madsen O., Desbois P., Pictet R., Jami J. (1989)—“β islet cells of pancreas are the site of expression of the human insulin gene in transgenic mice”; Exptl Cell Res. 180: 467-474.

Fromont-Racine M., Bucchini D., Madsen O., Desbois P., Linde S., Nielson J. H., Ripoche M- A., Jami J., Pictet R. (1990)—“Effect of 5′-flanking sequence deletions on expression of the human insulin gene in transgenic mice”; Mol. Endocrinol. 4: 669-677.

The human transgene was placed on a consanguine genetic background C57B1/6 by a series of 12 backcrosses with C57B1/6 animals.

The self-crossing of these heterozygous transgenic mice made it possible to obtain a line of mice which are homozygous for the transgene.

These transgenic mice Insh were crossed with mice described previously, in which one copy of the Ins2 gene and both copies of the Ins1 gene are invalidated.

In the descendants, the individuals which have an invalidated copy of Ins1 and of Ins2 and a copy of the human transgene were identified by DNA analysis.

Self-crossing between these triple-heterozygous mice made it possible to isolate, in their descendants, individuals which were homozygous for the three mutations, i.e. individuals in which both the Ins1 copies and both the Ins2 copies are invalidated, which are homozygous for LacZ at the Ins2 site and for the Insh transgene on chromosome No. 18.

These triple homozygote mices are not diabetic, become adult and are capable of reproducing.

They can serve as starting material for developing mouse β cell lines in which all the insulin produced is human insulin.

They can also be used for crossing with non-mutant mice in order to recover individuals bearing, in the heterozygous state, the Ins1, Ins2 mutation or the human transgene, it being possible for the individuals of each of the three types to be self-crossed separately in order again to obtain three stable and viable homozygous mouse lines for each of the mutations or for the transgene.

References:

1. Sambrook, J., Fritsch, E. F. and Maniatis, T. (1989). Molecular Cloning, A Laboratory Manual (Cold Spring Harbor Lab. Press, Plainview, N.Y.), 2nd Ed.

2. Bradley, A., Evans, M., Kaufman, M. H. and Robertson, E. J. (1984). Nature 309, 255-256.

3. Mansour, S. L., Thomas, K. R. and Capecchi, M. R. (1988). Nature 336, 348-352.

4. Joyner, A. L. (Ed.) Gene Targeting. A Practical Approach (IRL Press at Oxford University Press, UK).

5. Deltour, L., Jami J. and Bucchini D. (1992). Methods in Mol. Cell Biol. 3, 35-38.

6. Michalova, K., Bucchini, D., Ripoche M- A., Pictet R. and Jami, J. (1988). Hum. Genet. 80, 247-252.

7. Hanahan, D. (1985). Nature 315, 115-122.

8. Efrat, S., Leiser, M., Surana, M., Tal, M., Fusco-Demane, D. and Fleischer, N. (1993). Diabetes 42, 901-907.

9. Knaack, D., Fiore, D. M., Surana, M., Leiser, M., Laurance, M., Fusco-de Mane, D., Hegre, O.D., Fleischer, N. and Efrat, S. (1994). Diabetes 43, 1413-1417.

10. Efrat, S., Fusco-de Mane, D., Lemberg, H., al-Emran, O. and Wang, X. (1995). Proc. Natl Acad. Sci. USA 92, 3576-3580.

11. Lanza, R. P., Hayes, J. L. and Chick, W. L. (1996). Nature Biotechnology 14, 1107-1111.

1. Use of a non-human transgenic mammal in which at least one of the alleles for at least one of the two genes coding for endogenous insulin is rendered functionally inoperative with respect to the expression of insulin, for the determination of medicinal products which are active on pathologies involving insulin.

2. Use according to claim 1 of a non-human transgenic mammal in which the expression of endogenous insulin 1 and/or of endogenous insulin 2 is suppressed relative to normal expression, in particular in the cells of the pancreas.

3. Non-human transgenic mammal or mammalian cells in which at least one of the alleles for at least one of the two genes coding for endogenous insulin is rendered functionally inoperative with respect to the expression of insulin.

4. Non-human transgenic mammal or mammalian cells in which the expression of the gene coding for insulin 1 is suppressed.

5. Non-human transgenic mammal or mammalian cells in which the expression of the gene coding for insulin 2 is suppressed.

6. Non-human transgenic mammal or mammalian cells in which the expression of the gene coding for insulin 1 and that of the gene coding for insulin 2 are suppressed.

7. Non-human mammal or mammalian cells according to claim 3, in which at least one of the alleles coding for insulin 1 and/or insulin 2 is replaced with a gene capable of coding for a protein which has enzymatic activity.

8. Non-human transgenic mammal or mammalian cells in which the expression of the endogenous gene coding for insulin 1 and that of the endogenous gene coding for insulin 2 are suppressed, and which contains a transgene for expressing human insulin.

9. Non-human transgenic mammal or mammalian cells, in particular β cells, in which the expression of the endogenous gene coding for insulin 1 and that of the endogenous gene coding for insulin 2 are suppressed, and which also contains, on the one hand, a transgene for expressing human insulin, and, on the other hand, a transgene comprising the sequence coding for the T antigen of the SV40 virus under the control of the rat insulin 2 gene promoter, and which allows induction of the proliferation of the β cells.

10. Non-human transgenic mammal or mammalian cells, in particular β cells, in which the expression of the endogenous gene coding for insulin 1 and that of the endogenous gene coding for insulin 2 are suppressed, and which, in addition, contains, on the one hand, a transgene for expressing exogenous human insulin, and, on the other hand, a transgene comprising the sequence coding for the T antigen of the SV40 virus under the control of the rat insulin 2 gene promoter which has been premodified so as to induce termination of its transcription under the effect of the presence of tetracycline, and allowing stoppage of the proliferation of the β cells in the presence of tetracycline.

11. Non-human transgenic mammal or mammalian cells, in particular β cells, in which the expression of the endogenous gene coding for insulin 1 and that of the endogenous gene coding for insulin 2 are suppressed, and which, in addition, contains a transgene comprising the sequence coding for the T antigen of the SV40 virus under the control of the rat insulin 2 gene promoter which has been premodified so as to induce termination of its transcription under the effect of the presence of tetracycline, and allowing stoppage of the proliferation of the β cells in the presence of tetracycline.

12. Non-human transgenic mammal or mammalian cells, in particular β cells, in which the expression of the endogenous gene coding for insulin 1 and that of the endogenous gene coding for insulin 2 are suppressed, and which also contains, on the one hand, a transgene for expressing human insulin, and, on the other hand, a construct containing the transgene comprising the sequence coding for the T antigen of the SV40 virus under the control of the rat insulin 2 gene promoter which has been premodified so as to induce its transcription under the effect of the presence of a substance, in particular an antibiotic, a hormone, a cytokine or a growth factor, and which allows induction of the proliferation of the β cells in the presence of the abovementioned substance.

13. β cells in which the expression of the gene coding for insulin 1 and that of the gene coding for insulin 2 are suppressed, containing a transgene as defined in claims 8 to 12, which β cells are encapsulated in an inert material which is capable of grafting these cells in vivo under conditions sheltered from the immune system.

14. Cells cultured from non-human transgenic animals according to any one of claims 3 to 13.

15. Process for obtaining a transgenic model for studying pathologies involving insulin and for treatment of these pathologies, comprising:
replacement of all or part of the gene coding for insulin 1 with the neo gene, and in particular replacement of the nucleotide fragment ranging from position −0.8 kilobase to position +0.687 kilobase (with reference to the transcript origin located at position +1), and in particular
replacement of at least one of the alleles for the endogenous gene coding for insulin 1, in cells, in particular mouse embryonic strain (ES) cells, with a construct as obtained in the following manner and denoted by pIns1^{neo},
sub-cloning of a 7.2 kb ApaI/XhoI fragment (corresponding to the flanking region downstream (at the 3′ end) of Ins1) into a plasmid pSK⁺ predigested with ApaI and XhoI, resulting in p4,
sub-cloning of another fragment BamHI/HindIII (corresponding to the region at the 5′ end of Ins1) into pSK⁺, giving pR1,
the vector used to manufacture the recombination vector being derived from pKS⁺, containing the neo gene (BamHI fragment originating from pMC1 POLA -Stratagene) at the BamHI site, and the tk gene (XhoI/HindIII fragment originating from pMCtk described in Liu et al., Cell, 1993, Vol. 75, 59-72) between the XhoI and HindIII sites and denoted by pNTK,
cloning of the NotI/PvuII fragment of pR1 corresponding to the fragment of homologous sequence at the 5″ end into pNTK between the NotI and XbaI sites, giving pR2, in which the 6.5 kb HindIII fragment of p4 (corresponding to the fragment of homologous sequence at the 3′ end) has been cloned into the HindIII site giving pIns1^{neo}, and/or
replacement of all or part of the sequence coding for the insulin 2 gene with a sequence coding for a protein with enzymatic activity, and in particular replacement of the nucleotide fragment ranging from position +21 to position +856 base-pairs (with reference to the transcript origin located at position +1), and, in particular,
replacement of the coding sequence of one or two alleles of the endogenous gene coding for insulin 2 with that of the LacZ gene of Escherichia coli in mouse embryonic strain (ES) cells, and in particular with a construct as obtained in the following way and denoted by pIns2^{Zneo}
sub-cloning of a 7 kb EcoRI fragment, corresponding to the flanking region at the 3′ end of the insulin 2 gene, into the EcoRI site of pSK⁺, giving p12,
destruction of an NsiI site present in the 7 kb insert of p12, giving p12ΔNsiI,
sub-cloning of a 5 kb XbaI fragment containing the insulin 2 gene also into pSK⁺, giving p13,
synthesis of the −950/+20 region of the insulin 2 gene by a polymerase chain reaction (PCR) using the following nucleotide primers: 5′-CGCTCTAGACCCTCCTCTTGCATTTCAAA-3′ and 5′-CGCATGCATGTAGCGGATCACTTAGGGT-3′ these primers bringing XbaI and NsiI sites into the PCR product obtained,
cloning of the PCR product upstream of the sequence coding for the LacZ gene into the pGN vector (Le Mouellic et al., 1990, PNAS, 87, 4712-4716) between the XbaI and NsiI sites,
destruction of the NsiI site, giving p15,
replacement of the XbaI/SfiI fragment of p15 with an XbaI/SfiI fragment originating from p13, giving p16,
modification of the pNTK vector as defined above by inserting an NsiI linker therein, into the HindIII site, giving p10,
cloning of the XbaI/XhoI fragment originating from p16 (containing both the 2.7 kb fragment of homologous sequence at the 5′ end and LacZ) into p10 between the XbaI and SpeI sites, giving p17,
cloning of the 5.5 kb SmaI/EcoRI fragment originating from p12ΔNsiI (corresponding to fragment of homologous sequence at the 3′ end) into the NsiI site of p17 using NsiI linkers and giving pIns2^{Zneo},
introduction of the abovementioned cells into embryos, in particular non-human mammalian blastocytes, in particular mouse blastocytes,
selection of male chimeric animals according to a criterion corresponding to the ES line,
crossing of the selected animals with mice, in particular C57BL/6 mice, giving animals which are heterozygous relative to one of the constructs as defined according to one of claims 3 to 12, and
optionally, crossing of two heterozygotes in order to obtain an animal which is homozygous relative to one of the constructs as defined according to one of claims 3 to 12,
optionally, crossing of homozygotes relative to each of the constructs defined above in order to obtain double-heterozygotes for each of the constructs,
optionally, crossing of the double-heterozygotes, giving, in particular, double-homozygous animals.

16. Process for screening medicinal products which are active on pathologies involving insulin, in particular diabetes, comprising the administration of a medicinal product to be tested to a transgenic non-human mammal or to transgenic non-human mammalian cells
containing, in the place of at least one of the alleles of the endogenous gene coding for insulin 2 , a sequence coding for a protein with enzymatic activity, and in particular the nucleotide fragment ranging from position +21 base-pairs to position +856 base-pairs, and in particular a construct pIns2^{Zneo} as defined according to claim 15, and
optionally containing, in the place of at least one of the alleles of the endogenous gene coding for insulin 1 , the neo gene and in particular the nucleotide fragment ranging from position −0.8 kilobase to position +0.856 kilobase, and in particular a construct pIns1^{neo} as defined according to claim 15,
determination of the β-galactosidase activity of the β cells.

17. Transgenic construct in which:
all or part of the sequence of an allele of the endogenous gene coding for insulin 1 is replaced with the neo gene, and in particular the nucleotide fragment ranging from position −0.8 kilobase to position +0.687 kilobase, and in particular at least one of the alleles of the endogenous gene coding for insulin 1 is replaced in cells, in particular mouse embryonic strain (ES) cells, with a construct as obtained in the following manner and denoted by: pIns1^{neo},
sub-clonig of a 7.2 kb ApaI/XhoI fragment (corresponding to the flanking region downstream (at the 3′ end) of Ins1) into a plasmid pSK⁺ which has been predigested with ApaI and XhoI, resulting in p4,
sub-cloning of another BamHI/HindIII fragment (corresponding to the region at the 5′ end of Ins1) into pSK⁺, giving pR1,
the vector used to manufacture the recombination vector being derived from pKS⁺, containing the neo gene (BamHI fragment originating from pMC1 POLA-Stratagene) at the BamHI site, and the tk gene (XhoI/HindIII fragment originating from pMCtk described in Liu et al., Cell, 1993, Vol. 75, 59-72) between the XhoI and HindIII sites and denoted by pNTK,
clonig of the NotI/PvuII fragment of pR1 corresponding to the fragment of homologous sequence at the 5′ end into pNTK between the NotI and XbaI sites, giving pR2, in which the 6.5 kb HindIII fragment of p4, corresponding to the fragment of homologous sequence at the 3′ end, has been cloned into the HindIII site, giving pIns1^{neo},
and/or in which all or part of the sequence of an allele of the endogenous gene coding for insulin 2 is replaced with a sequence coding for a protein with enzymatic activity, and in particular the nucleotide fragment ranging from position +21 to position +856, and in particular, the coding sequence of one or two alleles of the endogenous gene coding for insulin 2 is replaced with that of the LacZ gene of Escherichia coli in mouse embryonic strain (ES) cells, and in particular with a construct as obtained in the following manner and denoted by pIns2^{Zneo},
sub-cloning of a 7 kb EcoRI fragment, corresponding to the flanking region at the 3′ end of the insulin 2 gene, into the EcoRI site of pSK⁺, giving p12,
destruction of an NsiI site present in the 7 kb insert of p12, giving p12ΔNsiI,
sub-cloning of a 5 kb XbaI fragment containing the insulin 2 gene also into pSK⁺, giving p13,
synthesis of the −950/+20 region of the insulin 2 gene by a polymerase chain reaction (PCR) using the following nucleotide primers: 5′-CGCTCTAGACCCTCCTCTTGCATTTCAAA-3′ and 5′-CGCATGCATGTAGCGGATCACTTAGGGT-3′ these primers bringing XbaI and NsiI sites into the PCR product obtained,
cloning of the PCR product upstream of the sequence coding for the LacZ gene into the pGN vector (Le Mouellic et al., 1990, PNAS, 87, 4712-4716) between the XbaI and NsiI sites,
destruction of the NsiI site, giving p15,
replacement of the XbaI/SfiI fragment of p15 with an XbaI/SfiI fragment originating from p13, giving p16,
modification of the pNTK vector as defined above by inserting an NsiI linker therein, into the HindIII site, giving p10,
cloning of the XbaI/XhoI fragment originating from p16 (containing both the 2.7 kb fragment of homologous sequence at the 5′ end and LacZ) into p10 between the XbaI and SpeI sites, giving p17,
cloning of the 5.5 kb SmaI/EcoRI fragment originating from p12ΔNsiI (corresponding to fragment of homologous sequence at the 3′ end) into the NsiI site of p17 using NsiI linkers and giving pIns2^{Zneo}.

18. Genomic DNA of insulin 1 of the consanguine mouse line 129, characterised by the following restriction sites, with reference to the transcript origin located at position +1:
upstream of the site +1:
two PvuII sites (at −8.4 and −0.8 kilobases)
two BamIII sites (at −3.9 and 3.3 kilobases)
one HindIII site (at −8.3 kilobases)
one ApaI site (at −0.4 kilobases)
downstream of the site +1:
one SmaI site (at +388 base-pairs)
two PvuII sites (at +479 base-pairs and +8 kilobases)
two HindIII sites (at +687 base-pairs and +7.3 kilobases)
one XhoI site (at +7.8 kilobases).

19. Genomic DNA of insulin 2 of the consanguine mouse line 129, characterised by the following restriction sites, with reference to the transcript origin located at position +1:
upstream of the site +1:
one NsiI site (at −10.8 kilobases)
two EcoRI sites (at −5.4 kilobases and −455 base-pairs)
one XbaI site (at −2.7 kilobases)
one SfiI site (at −239 base-pairs)
downstream of the site +1:
two EcoRI sites (at +378 base-pairs and 7.9 kilobases)
one SmaI site (at +856 base-pairs)
one XbaI site (at +2.2 kilobases)
one NsiI site (at +4.2 kilobases)
one XhoI site (at +7 kilobases).
